(19) United States
(12) Patent Application Publication
(10) Pub. No.: US 2022/0162091 A1
(43) Pub. Date: May 26, 2022

(54) PURE WATER MANUFACTURING MANAGEMENT SYSTEM AND PURE WATER MANUFACTURING MANAGEMENT METHOD

(71) Applicant: MITSUBISHI HEAVY INDUSTRIES, LTD., Tokyo (JP)

(72) Inventors: Masaru CHIYOMARU, Tokyo (JP); Hiroaki MIKAWA, Tokyo (JP); Yuichi OKUZAKI, Tokyo (JP)

(21) Appl. No.: 17/618,222

(22) PCT Filed: Aug. 4, 2020
(86) PCT No.: PCT/JP2020/029829

§ 371 Date: Dec. 10, 2021

(30) Foreign Application Priority Data
Oct. 4, 2019 (JP) 2019-183622

(51) Int. Cl. C02F 1/00; C02F 1/42; G01N 33/18

(52) U.S. Cl. CPC C02F 1/008; C02F 1/42; C02F 1/006; G01N 33/18; C02F 2301/043; C02F 2201/005; C02F 2209/40; C02F 2209/42; C02F 2303/16

(57) ABSTRACT

The pure water manufacturing management system includes a pure water manufacturing device; an analysis device that inspects water quality; a first valve provided to first piping connected to the outlet side of the manufacturing device, the first valve controlling the amount of pure water supplied to a storage tank; second piping that branches from the first piping and is connected to the analysis device; and a control device, the control device controlling the analysis device during supply of the pure water to the storage tank, repeatedly performing water quality inspection of the pure water flowing in through the second piping, opening the first valve when, as a result of the water quality inspection, the water quality of the pure water satisfies a prescribed standard, and closing the first valve when the water quality of the pure water does not satisfy the prescribed standard.

### TECHNICAL FIELD

The present disclosure relates to a pure water manufacturing management system and a Pure water manufacturing management method.

The present application. claims priority based on Japanese Patent Application. No. 2019-183622 filed in Japan on Oct. 4, 2019, the contents of which are incorporated herein by reference.

### BACKGROUND ART

In a line for performing a. surface treatment of aircraft members such as aluminum or titanium, there are treatment steps such as degreasing, washing, etching, and coating. For example, in a washing treatment step, a. product may be washed by using pure water. For example, the pure water is manufactured by sending water, from which impurities or organic substances are removed by making the water to pass through a sand filtration tower and an activated carbon tower, to an ion exchange resin device and by removing sodium, chloride, silica, or the like in the water using the ion exchange resin device. Since silica is a weak ion, it has the property of being difficult to adsorb to the ion exchange resin. Therefore, even when the ion exchange resin once adsorbs silica, it may then adsorb strong ions and release the silica instead. In this case, the pure water manufactured by passing through the ion exchange resin contains a large amount of silica, and the quality of the pure water deteriorates. When such a phenomenon occurs, it is necessary to perform a regeneration treatment of the ion exchange resin. In the related art, the water quality inspection of pure water (for example, the amount of content of silica) is often performed manually.

PTL 1 discloses a method for measuring the alkalinity of each substance in treatment liquid in a surface washing step of a semiconductor substrate. In the measuring method of PTL 1, a light emitting substance that chemically emits light in response to a specific substance in the treatment liquid is added to the treatment liquid to emit light, and the alkalinity of the specific substance is measured based on the light emitting lightness.

### CITATION LIST

### Patent Literature

[PTL 1] Japanese Unexamined Patent Application Publication Nb. 7-306146

### SUMMARY OF INVENTION

### Technical Problem

However, manual water quality inspection takes time, and it is difficult to consecutively monitor water quality by continuously repeating the water quality inspection. Therefore, there is a possibility that changes in water quality such as a rapid increase silica cannot be detected promptly.

The present disclosure provides a pure water manufacturing management system and a gore water manufacturing management method capable of solving the above problems.

### Solution to Problem

According to the present disclosure, there is provided a pure water manufacturing management system including: a manufacturing apparatus of pure water; an analysis device that inspects a water quality; a first valve that is provided on a first pipe connected to an outlet side of the manufacturing apparatus and controls an amount of the pure water supplied to a water storage tank; a second pipe that branches from the first pipe and is connected to the analysis device; and a control device, in which the control device controls the analysis device to repeatedly perform a water quality inspection of the pure water, which flows in through the second pipe and is manufactured by the manufacturing apparatus, while supply of the pure water from the manufacturing apparatus to the water storage tank is performed, opens the first valve when a water quality of the pure water meets a predetermined reference as a result of the water quality inspection, and closes the first valve when the water quality of the pure water does not meet the predetermined reference as a result of the water quality inspection.

According to the present disclosure, there is provided a pure water manufacturing management method of a system including a manufacturing apparatus of pure water, an analysis device that inspects a water quality, a first valve that is provided on a first pipe connected to an outlet side of the manufacturing apparatus and controls an amount of the pure water supplied to a water storage tank, and a second pipe that branches from the first pipe and is connected to the analysis device, the method including: by a control device, controlling the analysis device to repeatedly perform a water quality inspection of the pure water, which flows in through the and second pipe and is manufactured by the manufacturing apparatus, while supply of the pure water from the manufacturing apparatus to the water storage tank is performed; opening the first valve when a water quality of the pure water meets a predetermined reference as a result of the water quality inspection; and closing the first valve when the water quality of the pure water does not meet the predetermined reference as a result of the water quality inspection.

### Advantageous Effects of Invention

According to the pure water manufacturing management system and the pure water manufacturing management method described above, it is possible to automate water quality management in a pure water manufacturing process.

### BRIEF DESCRIPTION OF DRAWINGS

FIG. 1 is a configuration diagram showing an example of a pure water manufacturing management system according to an embodiment.

FIG. 2 is a configuration diagram showing an example of a continuous analysis device according to the embodiment.

FIG. 3 is a flowchart showing an example of a pure water manufacturing treatment according to the embodiment.

FIG. 4 is a diagram showing an example of a hardware configuration of a control device according to the embodiment

### DESCRIPTION OF EMBODIMENTS

<Embodiment>

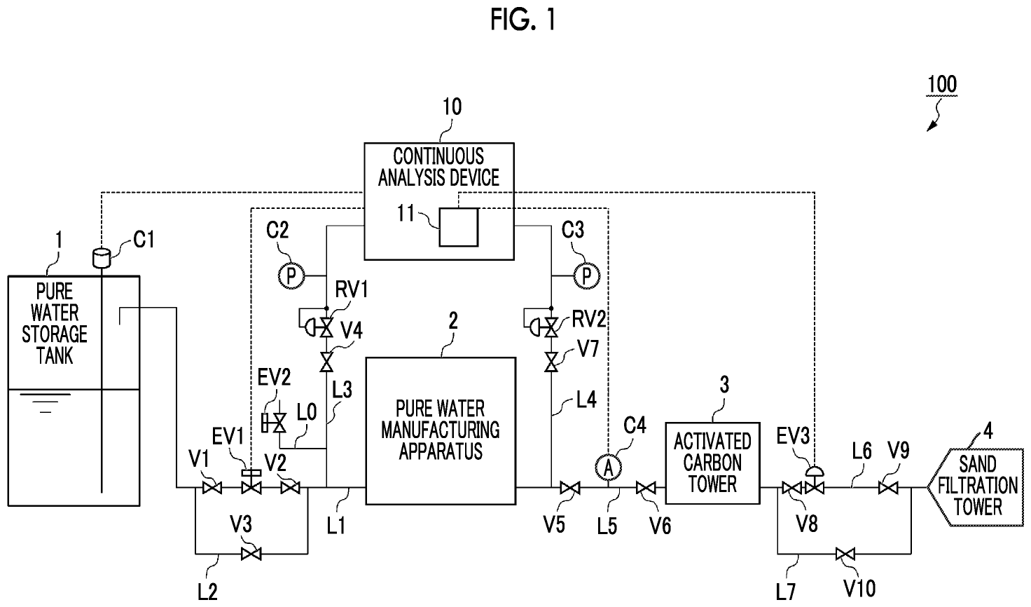

Hereinafter, a pure water manufacturing management system according to an embodiment will be described with reference to FIGS. 1 to 4.

(System Configuration)

FIG. 1 is a configuration diagram showing an example of the pure water manufacturing management system according to the embodiment.

The pure water manufacturing management system 100 is configured to include a pure water storage tank 1, a pure water manufacturing apparatus 2, an activated carbon tower 3, a sand filtration tower 4, a continuous analysis device 10, pipes (L1 to L7) for connecting therebetween, valves (V1 to V10, EV1 to EV3, and RV1 to RV2) provided on the pipes, and measurers (C1 to C4). Water flows from the right side to the left side in the drawing, and pure water manufactured by the pure water manufacturing apparatus 2 is supplied to the pure water storage tank 1. Hereinafter, an upstream-side in a direction in which the water flows is simply referred. to as an upstream-side, and a downstream-side in a direction in which the water flows is simply referred to as a downstream-side. Tap water is supplied to the sand filtration. tower 4. The sand filtration tower 4 removes impurities contained in the tap water. When water, from which the impurities are removed, flows out from the sand filtration tower 4, the water is supplied to the activated carbon tower 3 provided on the downstream-side. The sand filtration tower 4 and the activated carbon tower 3 are connected to each other by a pipe L6. The pipe L6 is provided with. a manual valve V9, an electromagnetic valve EV3, and a manual valve V8 in the order from the upstream-side. The manual valves V8 and V9 are adjusted to a predetermined. opening degree. The electromagnetic valve EV3 is controlled an opening degree such that the water flowing into the activated carbon tower 3 has a predetermined flow rate based on a flow rate measured by using a flow meter C4 described later. On the upstream-side of the manual valve V9 and the downstream-side of the manual valve V8, the pipe L7 is connected so as to bypass the electromagnetic valve EV3. The pipe L7 is provided with a manual valve V10. During automatic operation, the manual valve V10 is closed. The water, which flows out from the sand filtration tower 4, flows into the activated carbon tower 3 through the pipe L6.

The activated carbon tower 3 removes organic substances contained in the water. When water, from which the organic substances are removed, flows out from the activated carbon tower 3, the water is supplied to the pure water manufacturing apparatus 2 provided on the downstream-side, and a part of the water is supplied to the continuous analysis device 10. The activated carbon tower 3 and the pure water manufacturing apparatus 2 are connected to each other by a pipe L5. The pipe L5 is provided with a manual valve V6, a flow meter C4, and a manual valve V5 in the order from the upstream-side. The manual valves V5 and V6 are adjusted to the predetermined opening degree. The flow meter C4 is connected to a digital indication regulator 11 included in the continuous analysis device 10. The flow meter C4 measures the flow rate of the water flowing through the pipe L5 and outputs the measured value to the digital indication regulator 11. The digital indication regulator 11 is set to adjust the opening degree of the electromagnetic valve EV3 such that the flow rate of the water on the outlet side of the activated carbon tower 3, which is measured by using the flow meter C4, becomes the predetermined flow rate. The pipe L4, through which the water supplied to the continuous analysis device 10 passes, branches on the downstream-side of the manual valve V5 in the pipe L5. The pipe L4 connects the continuous analysis device 10 to the downstream-side of the manual valve V5 of the pipe L5. The pipe L4 is provided with a manual valve V7, a pressure-reducing valve RV2, and a pressure sensor C3 in the order along the water flow direction. The manual valve V7 is adjusted to the predetermined opening degree, and the pressure-reducing valve RV2 adjusts the pressure of the water supplied to the continuous analysis device 10. The pressure sensor C3 measures the pressure of the water flowing through the pipe L4. A part of the water, which flows out from the activated carbon tower 3, is sent to the continuous analysis device 10 through the pipe L4. The continuous analysis device 10 inspects water quality of this water. For example, the continuous analysis device 10 measures the electric conductivity, the PH, and the silica concentration of the sent water. A value measured by the continuous analysis device 10 is stored as indicating the water quality of the water treated by the pure water manufacturing apparatus 2 and is used for comparison with the water quality of the pure water manufactured by the pure water manufacturing apparatus 2, prediction of a period until regeneration or exchange of the ion exchange resin, or the like.

Most of the water, which flows out from the activated carbon tower 3, is supplied to the pure water manufacturing apparatus 2 through the pipe L5. The pure water manufacturing apparatus 2 includes the ion exchange resin. The pure water manufacturing apparatus manufactures the pure water, from which sodium, hydrochloric acid, or the like is removed by using an ion exchange resin, and supplies the pure water to the pure water storage tank 1. The pure water manufacturing apparatus 2 and the pure water storage tank 1 are connected to each other by a pipe L1. The pipe L1 is provided with a manual valve V2, an electromagnetic valve EV1, and a manual valve V1 in the order from the upstream-side. The manual valves V1 and V2 are adjusted to the predetermined opening degree. The continuous analysis device 10 controls the opening and closing of the electromagnetic valve EV1. On the upstream-side of the manual valve V2 and the downstream-side of the manual valve V1 in the pipe L1, the pipe L2 is connected so as to bypass the electromagnetic valve EV1. The pipe L2 is provided with a manual valve V3. During the automatic operation, the manual valve V3 is closed. The pipe L3, through which the pure water supplied to the continuous analysis device 10 passes, branches on the upstream-side of the manual valve V2 in the pipe L1. The pipe L3 connects the continuous analysis device 10 to the upstream-side of the manual valve V2 of the pipe L1. The pipe L3 is provided with. a manual valve V4, a pressure-reducing valve RV1, and a pressure sensor C2 in the order along the water flow direction. The manual valve V4 is adjusted to the predetermined opening degree, and the pressure-reducing valve RV1 adjusts the pressure of the pure water supplied to the continuous analysis device 10. The pressure sensor C2 measures the pressure of the pure water flowing through the pipe L3. A part of the pure water, which flows out from the pure water manufacturing apparatus 2, is sent to the continuous analysis device 10 through the pipe L3. The continuous analysis device 10 inspects the water quality of the pure water (electric conductivity, PH, and silica concentration). A certain reference is set for the water quality of the pure water stored in the pure water storage tank 1. When the value measured by the continuous analysis device 10 does not meet this reference, continuous analysis device 10 controls the electromagnetic valve EV1 to be closed and stops the supply of the pure water to the pure water storage tank 1. The value measured. by he continuous analysis device 10 is stored as indicating the water quality of the pure water manufactured by the pure water manufacturing apparatus 2 and used for predicting the period until the regenerated or the exchanged of the ion exchange resin.

The pipe L0 branches on the upstream-side of the manual valve V4 in the pipe L3. The electromagnetic valve EV2 is provided on the pipe L0. The pipe L0 is connected to a waste liquid tank (not shown). The continuous analysis device 10 controls the opening and closing of the electromagnetic valve EV2. For example, when the manufacture of the pure water is started, the continuous analysis device 10 opens the electromagnetic valve EV2 and closes the electromagnetic valve EV1, and then starts the manufacture and supply of the pure water after discharging water and the like accumulated in the activated carbon tower 3 or the ion exchange resin, through the pipe L0.

A level meter C1 is provided in the pure water storage tank 1. The level meter C1 measures a water level (water level) of the pure water storage tank 1 and outputs the measured value to the continuous analysis device 10. When the water level, which is measured by using the level meter C1, is lowered and reaches a predetermined value (a first threshold value), the continuous analysis device 10 causes the electromagnetic valves EV1 to EV3 or various sensors built tn the continuous analysis device 10 to the flow of the pure water to start the manufacture of the pure water. When the water level, which is measured by using the level meter C1, is raised and reaches a predetermined value (a second threshold value), the continuous analysis device 10 stops the manufacture of the pure water. In the pure water manufacturing management system 100, the continuous analysis device 10 controls the electromagnetic valve EV1 and the like and performs an automatic operation such that the amount of pure water, which is stored in the pure water storage tank 1 and meets the water quality reference, is maintained within a predetermined range. Next, the continuous analysis device 10 will be described.

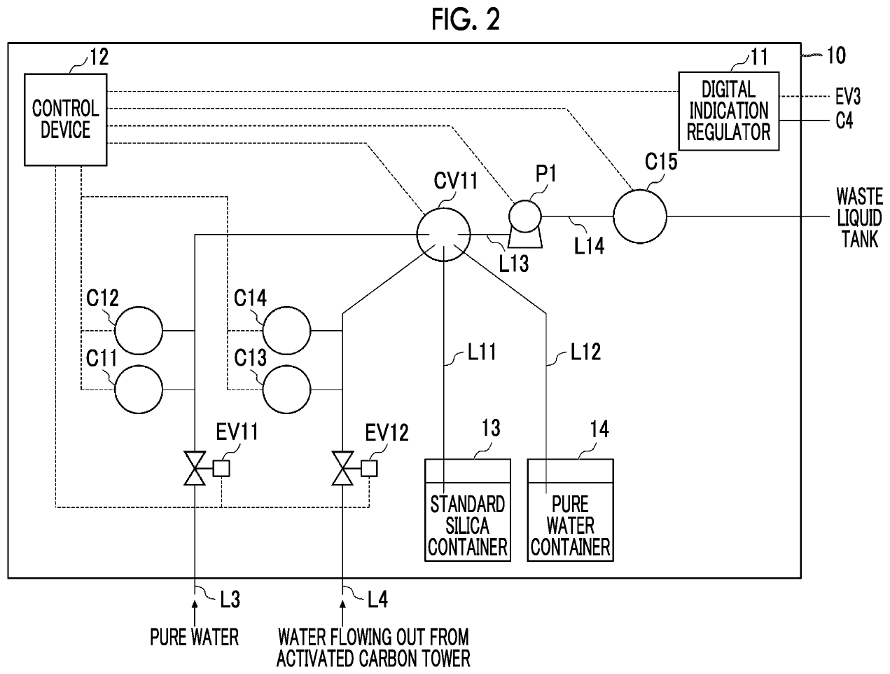

FIG. 2 is a configuration diagram showing an example of the continuous analysis device according to the embodiment.

FIG. 2 shows a schematic configuration diagram of the continuous analysis device 10. The continuous analysis device 10 includes a digital indication regulator 11, a control device 12, a standard silica container 13, a pure water container 14, a pump P1, a switching valve CV11, electromagnetic valves EV11 and EV12, and electric conductivity meters C11 and C13, PH meters C12 and C14, and silica concentration meter C15. The electric conductivity meter C11, PH meter C12, and the electromagnetic valve EV11 are provided on the pipe L3. The electric conductivity meter C13, the PH meter C14, and the electromagnetic valve EV12 are provided on the pipe L4. The pipes L3 and L4 are connected to the switching valve CV11. The standard silica container 13 and the switching valve CV11 are connected. with a pipe L11, and the pure water container 14 and the switching valve CV11 are connected with a pipe L12. The switching valve CV11 and the pump P1 are connected with. the pipe L13, and the pump P1 and the silica concentration meter C15 are connected with the pipe L14. A silica solution having a known concentration is stored in the standard silica container 13, and the pure water, in which the water quality inspection is performed, is stored in the pure water container 14. The silica concentration of the pure water in the pure water container 14 is known.

The digital indication regulator 11 adjusts the opening degree of the electromagnetic valve EV3 such that the flow rate, which is measured by using the flow meter C4 shown in FIG. 1, becomes a predetermined flow rate. The predetermined flow rate is a value set such that the amount of pure water supplied from the pure water manufacturing apparatus 2 to the pure water storage tank 1 becomes equal to or less than 5 tons/hour. In the related art, a case often occurs where the water level of the pure water storage tank 1 is visually checked and based on the check result, the opening degree of the valve corresponding to the electromagnetic valve EV3 is manually regulated to control the amount of pure water supply the pure water storage tank 1. In the present embodiment, the digital it regulator 11 can automatically adjust the flow rate of the water supplied to the pure water manufacturing apparatus 2 and the flow rate of the pure water supplied to the pure water storage tank 1.

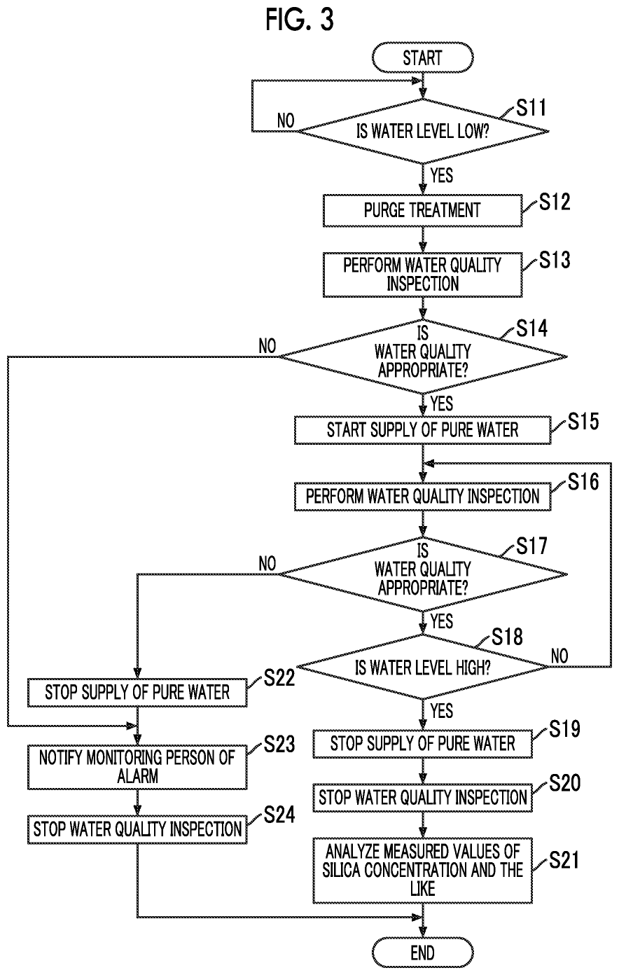

The control device 12 controls the pump P1 and the switching valve CV11 in addition to the electromagnetic valves EV1 to EV3 shown in FIG. 1 The control device 12 acquires measured values measured by the electric conductivity meters C11 and C13, the PH meters C12 and C14, and the silica concentration meter C15. The control device 12 analyzes the water quality and the like based on the measured values acquired from the silica concentration meter C15 and the like. The control device 12 is connected to notification. means such. as a monitor, a lamp, or a buzzer and notifies monitoring person of the occurrence of abnormalities through the notification means when there is the abnormality in water quality of the pure water or there is the abnormality in measurement accuracy of the silica concentration meter C15. The control of the electromagnetic valves EV1 to EV3 by the control device 12 will be described next with reference to FIG. 3. The inspection process of the water quality will be described with reference to FIG. 2.

(Water Quality Inspection Process)

For example, a part of the water, which flows out from the activated carbon tower 3, is sent to the continuous analysis device 10 through the pipe L4. When the water quality inspection of this water is performed, the control device 12 opens the electromagnetic valve EV12, closes the electromagnetic valve EV11, and controls the switching valve CV11 such that the pipe L4 and the pipe L13 communicate with each other. The control device 12 operates the pump P1. Thereafter, the water, which is sent to the pipe L4, passes through the electromagnetic valve EV12 of the pipe L4 and is sent to the electric conductivity meter C13 and the PH meter C14. The electric conductivity meter C13 measures the electric conductivity of the water and outputs the measured value to the control device 12. The PH meter C14 measures the PH of the water and outputs the measured value to the control device 12. Further, the water is sent to the silica concentration meter C15 via the switching valve CV11 and the pump P1. The silica concentration. meter C15 measures the silica concentration. of the water and outputs the measured value to the control device 12. The water used for the measurement is discharged as a waste liquid to the waste liquid tank (not shown).

The control device 12 performs the same control to perform the water quality inspection of the pure water manufactured by the pure water manufacturing apparatus 2. That is, the control device 12 opens the electromagnetic valve EV11, closes the electromagnetic valve EV12, and controls the switching valve CV11 such that the pipe L3 and the pipe L13 communicate with each other. The control device 12 operates the pump P1. Thereafter, the pure water, which is sent to the pipe L3, passes through the electromagnetic valve EV11 of the pipe L3 and is sent to the electric conductivity meter C11 and the PH meter C12. The electric conductivity meter C11 measures the electric conductivity of the pure water and outputs the measured value to the control device 12. The PH meter C12 measures the PH of the pure water and outputs the measured. value to the control device 12. The pure water is sent to the silica concentration meter C15 via the switching valve CV11 and the pump P1. The silica concentration meter C15 measures the silica concentration of the pure water and outputs the measured value to the control device 12. The pure water used for the measurement is discharged as a waste liquid to the waste liquid tank (not shown).

Further, the continuous analysis device 10 has a mechanism for inspecting the measurement accuracy of the silica concentration meter C15. For example, the continuous analysis device 10 supplies a standard silica solution or the pure water having a known silica concentration to the silica concentration meter C15 and inspects the measurement accuracy of the silica concentration meter C15 based on the measured values of the silica concentration meter C15 when those liquids are supplied.

For example, when the measurement accuracy of the silica concentration meter C15 is inspected. by using the standard silica solution, the control device 12 closes the electromagnetic valve EV11 and the electromagnetic valve EV12 and controls the switching valve CV11 such that the pipe L11 and the pipe L13 communicate with each other. Further, the control device 12 operates the pump P1. Thereafter, the standard silica solution in the standard silica container 13 is sucked, and the standard solution is sent to the silica concentration meter C15 via the switching valve CV11 and the pump P1. The silica concentration meter C15 measures the silica concentration in the standard silica solution and outputs the measured value to the control device 12. The standard silica solution used for the measurement is discharged as a waste liquid to the waste liquid tank (not shown). The control device 12 compares the measured value, which is measured by using the silica concentration meter C15, with the known silica concentration and determines whether or not a difference thereof is within a predetermined allowable range. When the difference is out of the allowable range, the control device 12 notifies the monitoring person of an abnormality in measurement accuracy of the silica concentration meter C15 by using the notification means.

For example, when the measurement accuracy of the silica concentration meter C15 is inspected by using the pure water, the control device 12 closes the electromagnetic valve EV11 and the electromagnetic valve EV12 and controls the switching valve CV11 such that the pipe L12 and the pipe L13 communicate with each other. Further, the control device 12 operates the pump P1. Thereafter, the pure water in the pure water container 14 is sucked and sent to the silica concentration meter C15 via the switching valve CV11 and the pump P1. The silica concentration meter C15 measures the silica concentration of the pure water and outputs the measured value to the control device 12. The pure water used for the measurement is discharged as a waste liquid to the waste liquid tank (not shown). The control device 12 compares the measured. value, which is measured by using the silica concentration meter C15, with the silica concentration (known) of the pure water and determines whether or not a difference thereof is within a predetermined allowable range. When the difference is out of the allowable range, the control device 12 notifies the monitoring person of an abnormality in measurement accuracy of the silica concentration meter C15 by using the notification means.

For example, with the water quality inspection of the water supplied through the pipe L4 and the water quality inspection of the pure water supplied through the pipe L3 as one set, the control device 12 may perform this water quality inspection repeatedly and continuously. Further, the control device 12 may continuously perform the water quality inspection of the pure water supplied through the pipe L3 and perform the water quality inspection of the water supplied through the pipe 14 at a longer time interval than the water quality inspection of the pure water. The control device 12 consecutively and continuously performs the water quality inspection of the pure water and the like and intermittently (for example, every day) performs a measurement accuracy inspection with the standard silica solution or the pure water. The switching of the liquid to be inspected can be smoothly switched the control of the control device 12 as described above. Therefore, the accuracy of the water quality inspection can be maintained by performing the measurement accuracy inspection of the silica concentration meter C15 without hindering the manufacture of the pure water. When the measurement accuracy of the silica concentration meter C15 is lowered, the accurate water quality inspection can be restarted by performing the maintenance promptly.

(Pure Water Manufacturing Treatment)

Next, an example of control of the pure water manufacturing management system 100 will be described with reference to FIG. 3.

FIG. 3 is a flowchart showing an example of the pure water manufacturing treatment according to the embodiment.

As a premise, the control device 12 acquires the water level of the pure water measured by using the level meter C1 in FIG. 1 at predetermined time intervals, and the digital indication regulator 11 acquires the flow rate measured by using the flow meter C4 in FIG. 1 at the predetermined. time intervals. As an initial state, it is assumed that the pure water manufacturing management system 100 does not perform the manufacture of the pure water. At this time, the control device 12 controls the electromagnetic valves EV1 to EV3 to be closed. The control device 12 stops the water quality inspection process by the continuous analysis device 10. For example, the control device 12 stops the pump P1 (FIG. 2). The control device 12 performs the following treatments at a predetermined control cycle.

First, the control device 12 determines whether or not the water level of the pure water storage tank 1 is low based on the water level measured by using the level meter C1 (step S11). For example, the control device 12 determines that the water level is low when the latest water level acquired from the level meter C1 is equal to or lower than a predetermined first threshold value, and determines that the water level is not low when the latest water level is above the first threshold value. When the water level is not low (step S11: No), it is not necessary to manufacture the pure water because a sufficient amount of pure water is stored. in the pure water storage tank 1. The control device 12 repeats the treatment of step S11 while maintaining the initial state.

When the water level is low (step S11: Yes), the control device 12 starts the manufacture of the pure water. First, the control device 12 performs a purge treatment (step S12). The purge treatment is a treatment of discharging the water or the like accumulated in the pure water manufacturing management system 100. The control device 12 closes the electromagnetic valve EV1 and opens the electromagnetic valve EV2. The control device 12 instructs the digital indication regulator 11 to open the electromagnetic valve EV3. The digital indication regulator 11 opens the electromagnetic valve EV3. As a result, the water, which is supplied to the sand filtration tower 4, discharged to the waste liquid tank (not shown) through the pipe L6, the activated carbon tower 3, the pipe L5, the pure water manufacturing apparatus 2, the pipe L1, the pipe L3, and the pipe L0. In this process, the water accumulated in the pure water manufacturing apparatus 2, the activated carbon tower 3, and each pipe is discharged. when a predetermined time elapses, the control device 12 performs the water quality inspection before the pure water is supplied to the pure water storage tank 1 (step S13). The control device 12 closes the electromagnetic valve EV12 and opens the electromagnetic valve EV11. The control device 12 starts the water quality inspection. process by the continuous analysis device 10. For example, the control device 12 controls the switching valve CV11 to make the pipe L3 and the pipe L13 communicate with each other and activates the pump P1. As a result, the water, which is supplied to the sand filtration tower 4, passes through the pipe L6, the activated carbon tower 3, the pipe L5, the pure water manufacturing apparatus 2, the pipe L1, and the pipe L3, and is sent to the electric conductivity meter C11, the PH meter C12, and the silica concentration meter C15 included in the continuous analysis device 10. Each measurer measures the water quality and outputs the measurement result to the control device 12. The control device 12 compares the electric conductivity, the PH, and the silica concentration with the respective reference values to determine whether or not the water quality is appropriate. When a difference between each of the measured values and each of the reference values exceeds the predetermined allowable range, the control device 12 determines that the water quality is not appropriate. When a difference between each of the measured values and each of the reference values is within the predetermined allowable range, the control device 12 determines that the water quality is appropriate.

When it is determined that the water quality is not appropriate (step S14: No), the control device 12 notifies the monitoring person of an alarm that the water quality of the pure water manufactured by the pure water manufacturing apparatus 2 does not meet the reference, by using communication means (step S23). For example, the control device 12 displays he monitor a message recommending the regeneration at the ion exchange resin or the exchange of the ion exchange resin, as well as the electric conductivity, PH, and silica concentration of the pure water. Next, the control device 12 stops the water quality inspection (step S24). The control device 12 stops the pump P1 and closes the electromagnetic valve EV3.

When it is determined that the water quality is appropriate (step S14: Yes), the control device 12 starts the supply of the pure water to the pure water storage tank 1 (step S15). Specifically, the control device 12 controls the electromagnetic valve EV1 to be opened. The control device 12 instructs the digital indication regulator 11 to control the opening degree of the electromagnetic valve EV3. The digital indication regulator 11 controls the opening degree of the electromagnetic valve EV3 such that the flow rate, which is measured by using' the flow meter C4, becomes the predetermined flow rate. As a result, the water, which is supplied to the sand filtration tower 4, is adjusted to an appropriate flow rate by the electromagnetic valve EV3 and passes through the pipe L6, the activated carbon tower 3, the pipe L5, the pure water manufacturing apparatus 2, and the pipe L1 to supply to the pure water storage tank 1.

The control device 12 consecutively performs the water quality inspection of the pure water (step S16). For example, the control device 12 controls the switching valve CV11 so as to make the pipe L3 and the pipe L13 communicate with each other to activate the pump P1 only for a time during which the amount of pure water required for the water quality inspection can be taken in. When the pure water is taken in, the control device 12 stops the pump P1 until the inspection is ended. Next, when the predetermined time elapses, the pump P1 is activated again and the water quality inspection of the pure water is performed. Alternatively, the control device 12 may control the switching valve CV12 so as to make the pipe L4 and the pipe L13 communicate with each other and activate the pump P1 for a predetermined time to perform the water quality inspection of the water that passes through the activated carbon tower 3. As described with reference to FIG. 2, in the continuous analysis device 10, the water quality inspection can be performed by switching between the pure water manufactured by the pure water manufacturing apparatus 2 and the water treated by the pure water manufacturing apparatus 2. Further, the measurement accuracy inspection of the silica concentration meter C15 can be performed by selecting either the silica solution having a known concentration or the pure water in which the inspection is performed. Any treatment can be set as to which of these treatments to be performed. The monitoring person sets a cycle for performing the water quality inspection of the pure water and the water, and a cycle for performing the measurement accuracy inspection of the silica concentration meter C15, in the control device 12. The control device 12 switches the inspection target in accordance with the cycle set by the monitoring person, and repeatedly performs the water quality inspection and the like. For example, the control device 12 performs the water quality inspection of the pure water manufactured by the pure water manufacturing apparatus 2 once every 30 minutes. For example, the control device 12 performs the water quality inspection on both the pure water manufactured by the pure water manufacturing apparatus 2 and the water on the outlet side of the activated carbon tower 3, once every 30 minutes.

Each time the water quality inspection of the pure water is performed, the control device determines whether or not the water quality is appropriate in the same manner as in step S14 (step S17). When the water quality is not appropriate (step S17: No), the control device 12 closes the electromagnetic valve EV1 and stops the supply of the pure water to the pure water storage tank 1 (step S22). Next, the control device 12 notifies the monitoring person of an alarm that the water quality of the pure water does not meet the reference, by using the notification means (step S23). Next, the control device 12 stops the water quality inspection (step S24). The control device 12 stops the pump P1 and closes the electromagnetic valve EV3.

When the water quality is appropriate (step S17: Yes), the control device 12 determines whether the water level of the water storage tank is high (step S18). For example, the control device 12 determines that the water level is high when the latest water level acquired from the level meter C1 is equal to or higher than a predetermined second threshold value (second threshold value>first threshold value), and determines that the water level is not high when the latest water level is below the second threshold value. When the water level is not high (step S18: No), the control device 12 repeats the treatment from step S16 while consecutively supplying the pure water to the pure water storage tank 1.

When the water level is high (step S18: Yes), the control device 12 stops the supply of the pure water because a sufficient amount of pure water is stored in the pure water storage tank 1 (step S19). Specifically, the control device 12 closes the electromagnetic valve EV3 and then closes the electromagnetic valve EV1. The control device 12 stops the water quality inspection (step S20). The control device 12 stops the pump P1.

Next, the control device 12 analyzes the measured values of the electric conductivity, the PH, and the silica concentration (step S21). For example, the control device 12 analyzes a relationship between the elapsed time since the last time the ion exchange resin was regenerated or exchanged or the amount of manufactured pure water, and the measured values of the electric conductivity, the PH, and the silica concentration. Based on the trends of the electric conductivity, the PH, and the silica concentration, the control device 12 predicts the exchange time of the ion exchange resin and the like. For example, in three graphs where the horizontal axis is defined as time and the vertical axis is respectively defined as the electric conductivity, the PH, and the silica concentration, the control device 12 extrapolates each of the graphs of the electric conductivity, the PH, and the silica concentration and predicts the time when any of the values of electric conductivity, PH, and silica concentration exceeds the threshold value set for each as the exchange time of the ion exchange resin or the regeneration time of the ion exchange resin.

Similarly, the control device 12 may analyze changes in electric conductivity, PH, and silica concentration of the water collected on the outlet side of the activated carbon tower 3 and use the result for the prediction of the exchange time of the ion exchange resin and the like. For example, in a case where the water quality of the water, which is collected on the outlet side of the activated carbon tower 3, is worse than before, when extrapolating the graphs of the electric conductivity, the PH, and the silica concentration of the pure water illustrated above, the exchange time of the ion exchange resin may be predicted by increasing the inclination according to the degree of deterioration of the water quality. Alternatively, the control device 12 may predict the exchange time of the ion exchange resin and the like based on a difference between the result of the water quality inspection of the water collected on the outlet side of the activated carbon tower 3 and the result of the water quality inspection of the pure water manufactured by the pure water manufacturing apparatus 2. For example, when the difference between the two results in terms of the silica concentration is within the predetermined range, it may be determined that the adsorption capacity of silica is reduced, and the predetermined period ahead may be predicted as the exchange time of the ion exchange resin. Alternatively, the control device 12 may predict the exchange time of the ion exchange resin and the like based on a change in difference between the result of the water quality inspection of the water collected on the outlet side of the activated carbon tower 3 and the result of the water quality inspection of the pure water manufactured by the pure water manufacturing apparatus 2. For example, a difference between the difference of the two results in terms of the silica concentration immediately after the exchange of the ion exchange resin and the difference of the two results in terms of the latest silica concentration is calculated, and when the difference is larger than a predetermined value, it may be determined that the adsorption capacity of silica reduced, and the predetermined period ahead may be predicted as the exchange time of the ion exchange resin. The control device 12 notifies the monitoring person of the predicted exchange time or regeneration time of the ion exchange resin, by using the notification means.

In the related art, a case often occurs where the water level of the pure water storage tank 1 is visually checked and by manually regulating the valve corresponding to the electromagnetic valve EV3 in FIG. 1, the amount of water feeding is adjusted to manufacture the pure water, and then the water quality inspection is performed manually for the manufactured pure water. In contrast to this, according to the pure water manufacturing management system 100 of the present embodiment, the water quality of the pure water manufactured by the pure water manufacturing apparatus 2 can be continuously and consecutively monitored. As a result, even when the water quality of the pure water suddenly changes due to deterioration of the ion exchange resin or the like, the change can be quickly detected and handled. The change in water level in the pure water storage tank 1 is monitored, the manufacture of the pure water is automatically started when the amount of pure water stored is insufficient, and the manufacture of the pure water is automatically stopped when the amount of pure water stored. reaches the maximum value. By introducing the digital indication regulator 11, the speed of supplying the pure water to the pure water storage tank 1 can be automatically controlled to an appropriate amount according to the performance of the pure water manufacturing apparatus 2 and the like. With these functions, according to the present embodiment, it is possible to automate the manufacture of the pure water in which quality is ensured.

In the pure water manufacturing management system 100 illustrated in FIG. 1, it is possible to automate only the water quality inspection while manually controlling the manufacture of the pure water as in the related art. Specifically, when the measured value of the level meter C1 becomes a low water level, a user closes the manual valves V1, V2, V3, V8, and V9. Thereafter, the electromagnetic valve EV2 and the manual valve V10 are opened. When the purge treatment is performed while maintaining this state for a predetermined time, the electromagnetic valve EV2 is closed and the control device 12 is instructed to start the water quality inspection.

In the same manner as in step S13 in FIG. 3, the control device 12 performs the water quality inspection of the pure water, which is manufactured by the pure water manufacturing apparatus 2, and notifies the user of the inspection result by using the notification means. When the water quality appropriate, the user opens the manual valve V3. The user regulates the opening degree of the manual valve V10 such that the amount of pure water supplied to the pure water storage tank 1 is, for example, equal to or less than 5 tons/hour while observing the measured value of the flow meter C4. As a result, for example, even when the electromagnetic valves EV1 and EV3 fail, the pure water manufacturing management system 100 can be manually operated, and it is possible to manufacture the pure water in which quality is ensured.

As described above, the measurement and the management of the silica concentration are aimed at the manufacture of the pure water and the application to the water quality management, but in addition, it can also be applied to washing water of treatment liquid in an aircraft surface treatment line and managing the silica concentration of treatment liquid (for example, chromate treatment liquid).

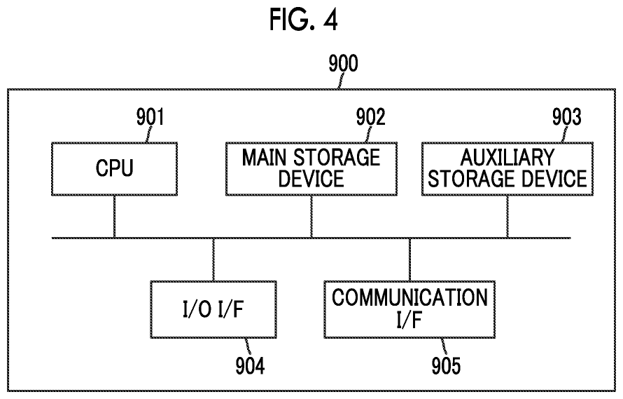

FIG. 4 is a diagram showing an example of a hardware configuration of the control device according to the embodiment.

A computer 900 includes a CPU 901, a main storage device 902, an auxiliary storage device 903, an I/O interface 904, and a communication interface 905.

The control device 12 described above is mounted on the computer 900. Further, the above-mentioned each function is stored in the auxiliary storage device 903 in the form of a program. The CPU 901 reads the program from the auxiliary storage device 903, loads the program into the main storage device 902, and executes the above treatments according to the program. The CPU 901 ensures a storage area in the main storage device 902 according to the program. The CPU 901 ensures a storage area for storing the data being processed in the auxiliary storage device 903 according to the program.

A program for implementing all or a part of the functions of the control device 12 may be recorded on a computer-readable recording medium, and the program recorded on the recording medium may be read by a computer system and executed to perform processes by each functional unit. The term “computer system” as used herein includes hardware such as an OS or peripheral devices. The “computer system” is also assumed to include a homepage providing environment (or display environment) when a WWW system used. The “computer-readable recording medium” refers to a portable medium such as a CD, DVD, or USB, or a storage device such as a hard disk built in the computer system. When this program is distributed to the computer 900 by using a communication line, the computer 900, which is received the distribution of the program, may load the program into the main storage device 902 and execute the above processes. The above-mentioned program may be a program for implementing a part of the above-mentioned. functions and further implementing the above-mentioned functions in combination with a program already recorded in the computer system.

As described above, some embodiments according to the present disclosure have been described, but all of these embodiments are presented as examples and are not intended to limit the scope of the invention. These embodiments can be implemented in various other forms, and. various omissions, replacements, and changes can be made without departing from the gist of the invention. These embodiments and variations thereof are included in the scope of the invention described in the claims and the equivalent scope thereof, as are included in the scope and gist of the invention.

<Additional Notes>

The pure water manufacturing management system. 100 and the pure water manufacturing management method described in each embodiment are ascertained as follows, for example.

(1) According to a first aspect, there is provided a pure water manufacturing management system. (100) including: a manufacturing apparatus (2) of pure water; an analysis device (10) that inspects a water quality; a first valve (EV1) that is provided on a first pipe (L1) connected to an outlet side of the manufacturing apparatus and controls an amount of the pore water supplied to a water storage tank; a second pipe (L3) that branches from the first pipe (L1) and is connected to the analysis device; and a control device (12), in which the control device (12) controls the analysis device (10) to perform a water quality inspection of the pure water, which flows in through the second pipe (L3) and is manufactured by the manufacturing apparatus (2), while supply of the pure water from the manufacturing apparatus (2) to the water storage tank (1) is performed, opens the first valve (EV1) when a water quality of the pure water meets a predetermined reference as a result of the water quality inspection, and closes the first valve when the water quality of the pure water does not meet the predetermined reference as a result of the water quality inspection.

According to the pure water manufacturing management system (100), in parallel while supplying the pure water, the water quality inspection performed for the manufactured pure water and the pure water is consecutively supplied when the water quality meets the reference. As a result, the quality of the pure water supplied to the water storage tank (1) can be ensured.

The water quality inspection can be continuously performed by controlling the control device (12).

(2) The pure water manufacturing management system (100) according a second aspect is the pure water manufacturing management system of (1), the system further includes a third valve (EV3) that is provided on third pipes (L5, L6) connected to an inlet side of the manufacturing apparatus (2) and controls an amount of water supplied to the manufacturing apparatus (2), in which the control device (12) closes the first valve (EV1), opens the third valve (EV3), and activates the analysis device (10) to start the water quality inspection of the pure water flowing in through the second pipe (L3), when the manufacturing apparatus (2) starts the supply of the pure water to the water storage tank (1), and opens the first valve when the water quality of the pure water meets the predetermined reference as a result of the water quality inspection.

As a result, when the supply of the pure water is started, the water quality of the pure water manufactured by the manufacturing apparatus (2) is inspected and the supply of the pure water is started when the water quality meets the reference. As a result, the water quality of the pure water supplied to the water storage tank (1) can be ensured.

(3) The pure water manufacturing management system (100) according a third aspect is the pure water manufacturing management system of (2), the system further includes a second valve (EV2) that is provided on a discharge pipe (L0) connected to the outlet side of the manufacturing apparatus (2), in which the control device (12) opens the third valve (EV3) and opens the second valve (EV2) when the manufacturing apparatus (2) starts the supply of the pure water to the water storage tank (1), closes the second valve (EV2) after a predetermined time elapses, and thereafter starts the water quality inspection by the analysis device (10).

As a result, since the water quality inspection of the pure water, which is manufactured by the manufacturing apparatus (2), can be performed after discharging the water, which is accumulated in the manufacturing apparatus (2), before the start of the supply of the pure water, accurate water quality inspection can be performed.

(4) The pure water manufacturing management system (100) according to a fourth aspect is the pure water manufacturing management system of (2) to (3), in which the control device (12) controls an opening degree of the third valve (EV3) such that a flow rate of water supplied from the third pipes (L5, L6) to the manufacturing apparatus (2) becomes a predetermined flow rate.

As result, it is possible to automate the water feeding control of the water supplied to the manufacturing apparatus (2) and the pure water supplied to the water storage tank (1).

(5) Pure water manufacturing management system (100) according a fifth aspect is the pure water manufacturing management system of (2) to (4), the system further includes a fourth pipe (L4) that branches from the third pipes (L5, L6) and is connected to the analysis device (10), in which the analysis device (10) performs the water quality inspection of water flowing in through the fourth pipe (L4).

As a result, the water quality inspection of the water flowing into the manufacturing apparatus (2) can be performed. By performing the water quality inspection of the water flowing into the manufacturing apparatus (2), the water quality of the water that requires treatment by the manufacturing apparatus (2) can be ascertained, and the water quality of the pure water manufactured. by the manufacturing apparatus (2) from the water can be compared.

(6) The pure water manufacturing management system (100) according to a sixth aspect the pure water manufacturing management system of (1) to (5), in which the control device (12) starts the supply of the pure water when a water level of the water storage tank (1) measured by using a level meter becomes equal to or lower than a predetermined first threshold value, and closes the first valve to stop the supply of the pure water when the water level becomes equal to or higher than a predetermined second threshold value.

As a result, it is possible to automate the control of the start and end of the supply of the pure water.

(7) The pure water manufacturing management system (100) according to seventh aspect is the pure water manufacturing management system of (1) to (6), in which the control device (12) issues an alarm when the water quality of the pure water does not meet the predetermined reference.

As a result, it is possible to ascertain that an abnormality occurs in the quality of the pure water.

(8) The pure water manufacturing management system (100) according to an eighth aspect is the pure water manufacturing management system of (1) to (7), in which the control device (12) analyzes the result of the water quality inspection, and predicts at least one of a regeneration time and an exchange time of an ion exchange resin included in the manufacturing apparatus (2).

As a result, new ion exchange resin can be prepared in advance, and an implementation plan or an exchange plan for the regeneration treatment of the ion exchange resin can be proposed.

(9) The pure water manufacturing management system (100) according to ninth aspect is the pure water manufacturing management system of (2), the system further includes: a first bypass pipe (L2) that bypasses the first valve (EV1); a first manual valve (V3) that is provided on the first bypass pipe (L2); a third bypass pipe (L7) that bypasses the third valve (EV3); and a third manual valve (V10) that is provided on the third bypass pipe (L7).

As a result, even when. the first valve (EV1) and the third valve (EV3) are closed, the pure water can be supplied using the first bypass pipe, and the water can be supplied to the manufacturing apparatus using the third bypass pipe.

(10) According to tenth aspect, there is provided a pure water manufacturing management method of a system including a manufacturing apparatus of pure water, an analysis device that inspects a water quality, a first valve that is provided. on a first pipe connected to an outlet side of the manufacturing apparatus and controls an amount of the pure water supplied to a water storage tank, and a second pipe that branches from the first pipe and connected to the analysis device, the method including: by a control device, controlling the analysis device to repeatedly perform a water quality inspection of the pure water, which flows in through the second pipe and is manufactured by the manufacturing apparatus, while supply of the pure water from the manufacturing apparatus to the water storage tank is performed; opening the first valve when a water quality of the pure water meets a predetermined reference as a result of the water quality inspection; and closing the first valve when the water quality of the pure water does not meet the predetermined reference as a result of the water quality inspection.

### REFERENCE SIGNS LIST

100 pure water manufacturing management system.

1 pure water storage tank

2 pure water manufacturing apparatus

3 activated carbon tower

4 sand filtration tower

10 continuous analysis device

11 digital indication regulator

12 control device

13 standard silica container

14 pure water container

P1 pump

CV11 switching valve

EV1, EV2, EV3, EV11, EV12 electromagnetic valve

C11, C13 electric conductivity meter

C12, C14 PH meter

C15 silica concentration meter

V1 to V10 manual valve

RV1 to RV2 pressure-reducing valve

L1 to L7 pipe

C1 level meter

C2, C3 pressure sensor

C4 flow meter

900 computer

901 CPU

902 main storage device

903 auxiliary storage device

904 I/O interface

905 communication interface

1. A pure water manufacturing management system comprising:
a manufacturing apparatus of pure water;
an analysis device that inspects a water quality;
a first valve that is provided on a first pipe connected to an outlet side of the manufacturing apparatus and controls an amount of the pure water supplied to a water storage tank;
a second pipe that branches from the first pipe and is connected to the analysis device; and
a control device, wherein
the control device
controls the analysis device to repeatedly perform a water quality inspection of the pure water, which flows in through the second pipe and is manufactured by the manufacturing apparatus, while supply of the pure water from the manufacturing apparatus to the water storage tank is performed,
opens the first valve when a water quality of the pure water meets a predetermined reference as a result of the water quality inspection, and
closes the first valve when the water quality of the pure water does not meet the predetermined reference as a result of the water quality inspection.

2. The pure water manufacturing management system according to claim 1, further comprising:
a third valve that is provided on a third pipe connected to an inlet side of the manufacturing apparatus and controls an amount of water supplied to the manufacturing apparatus, wherein
the control device
closes the first valve, opens the third valve, and activates the analysis device to start the water quality inspection of the pure water flowing in through the second pipe, when the manufacturing apparatus starts the supply of the pure water to the water storage tank, and
opens the first valve when the water quality of the pure water meets the predetermined reference as a result of the water quality inspection.

3. The pure water manufacturing management system according to claim 2, further comprising:
a second valve that is provided on a discharge pipe connected to the outlet side of the manufacturing apparatus, wherein
the control device
opens the third valve and opens the second valve when the manufacturing apparatus starts the supply of the pure water to the water storage tank,
closes the second valve after a predetermined time elapses, and thereafter
starts the water quality inspection by the analysis device.

4. The pure water manufacturing management system according to claim 2, wherein
the control device controls an opening degree of the third valve such that a flow rate of water supplied from the third pipe to the manufacturing apparatus becomes a predetermined flow rate.

5. The pure water manufacturing management system according to claim 2, further comprising:
a fourth pipe that branches from the third pipe and is connected to the analysis device, wherein
the analysis device performs the water quality inspection of water flowing in through the fourth pipe.

6. The pure water manufacturing management system according to claim 1, wherein
the control device
starts manufacture of the pure water when a water level of the water storage tank measured by using a level meter becomes equal to or lower than a predetermined first threshold value, and
closes the first valve when the water level becomes equal to or higher than a predetermined second threshold value.

7. The pure water manufacturing management system according to claim 1, wherein
the control device issues an alarm when the water quality of the pure water does not meet the predetermined reference.

8. The pure water manufacturing management system according to claim 1, wherein
the control device
analyzes the result of the water quality inspection, and
predicts at least one of a regeneration time and an exchange time of an ion exchange resin included in the manufacturing apparatus.

9. The pure water manufacturing management system according to claim 2, further comprising:
a first bypass pipe that bypasses the first valve;
a first manual valve that is provided on the first bypass pipe;
a third bypass pipe that bypasses the third valve; and
a third manual valve that is provided on the third bypass pipe.

10. A pure water manufacturing management method of a system including
a manufacturing apparatus of pure water,
an analysis device that inspects a water quality,
a first valve that is provided on a first pipe connected to an outlet side of the manufacturing apparatus and controls an amount of the pure water supplied to a water storage tank, and
a second pipe that branches from the first pipe and is connected to the analysis device, the method comprising:
by a control device,
controlling the analysis device to repeatedly perform a water quality inspection of the pure water, which flows in through the second pipe and is manufactured by the manufacturing apparatus, while supply of the pure water from the manufacturing apparatus to the water storage tank is performed;
opening the first valve when a water quality of the pure water meets a predetermined reference as a result of the water quality inspection; and
closing the first valve when the water quality of the pure water does not meet the predetermined reference as a result of the water quality inspection.
